(19) United States
(12) Patent Application Publication
(10) Pub. No.: US 2025/0135139 A1
(43) Pub. Date: May 1, 2025

(54) RESPIRATION INDICATING DEVICE

(71) Applicant: PEMDX PTY LTD, Queensland (AU)

(72) Inventors: Phillip MELKSHAM, Queensland (AU); Mariam ROWE, Queensland (AU); Dylan Ray ROWE, Queensland (AU)

(73) Assignee: PEMDX PTY LTD, Queensland (AU)

(21) Appl. No.: 18/835,507

(22) PCT Filed: Feb. 3, 2023
(86) PCT No.: PCT/AU2023/050070

§ 371 Date: Aug. 2, 2024

(30) Foreign Application Priority Data
Feb. 3, 2022 (AU) 2022900204

(51) Int. Cl. A61M 16/06; A61M 16/00

(52) U.S. Cl. CPC A61M 16/06; A61M 16/0003; A61M 2016/0015; A61M 2202/0007; A61M 2202/0208; A61M 2205/0216; A61M 2205/025; A61M 2205/583

(57) ABSTRACT

A respiration indicating device configured to attach directly to an exhalation port of an oxygen mask, the respiration indicating device comprising a collapsible open-ended bag having a first opening at a first end and a second opening at a second end opposite the first end.

### TECHNICAL FIELD

The present invention relates to a respiration indicating device and method.

### BACKGROUND

Any references to methods, apparatus or documents of the prior art are not to be taken as constituting any evidence or admission that they formed, or form part of the common general knowledge.

The monitoring of human respiration in conscious, sedated and unconscious patients has long been a perplexing problem that has, in some ways, been overcome by advances in technology.

The medical profession has devised a number of techniques to monitor and determine respiration and respiratory qualities of a patient. Some of these techniques include physically observing chest excursion or chest auscultation, misting on a mirror and movement of air flow from the nasal passages. However, these techniques can be prone to human error and may produce unreliable results.

Some more advanced monitoring techniques include measuring the amount of carbon dioxide (CO₂) expired from respiratory gases. The measurement of carbon dioxide in expired respiratory gases is a known method for use with intubated patients (i.e. patients having a tube placed into the trachea).

However, during the perioperative period, the monitoring of CO₂ in respiratory gases from patients is often not undertaken, typically for logistical and resource related reasons. Instead, during this time, the respiratory monitoring and breath detection is conducted by the less reliable practices of observation and examination described above.

### SUMMARY OF INVENTION

In an aspect, the invention provides a respiration indicating device configured to attach to an exhalation port of an oxygen mask, the respiration indicating device comprising:
a collapsible open-ended bag having a first opening at a first end and a second opening at a second end opposite the first end,
wherein during inhalation, gases from a surrounding atmosphere flow into the bag from the second end to the first end and wherein during exhalation, exhaled gases flow into the bag from the first end to the second end, such that during both inhalation and exhalation, the bag is at least partially inflated by movement of gases through the bag.

Preferably, the respiration indicating device is configured to attach directly to an exhalation port of an oxygen mask

Preferably, the collapsible open-ended bag is configured to attach to an exhalation port of an oxygen mask. Preferably, the collapsible open-ended bag is configured to attach directly to an exhalation port of an oxygen mask.

In another aspect, the invention provides a method for indicating respiration, the method comprising:
attaching a collapsible open-ended bag having a first opening at a first end and a second opening at a second end opposite the first end to an oxygen mask, wherein the first opening at least partially covers an exhalation port of the oxygen mask;
during inhalation, gases from a surrounding atmosphere flow into the bag from the second end to the first end; and
during exhalation, exhaled gases flow from the oxygen mask into the bag from the first end to the second end, such that during both inhalation and exhalation, the bag is at least partially inflated by movement of gases through the bag.

In another aspect, the invention provides a respiration indicating system comprising:
an oxygen mask having an exhalation port;
a collapsible open-ended bag having a first opening at a first end and a second opening at a second end opposite the first end, the first end attached to the oxygen mask over a ventilation port such that an interior of the bag receives exhaled gases from within the oxygen mask,
wherein, in use, during inhalation, gases from a surrounding atmosphere flow into the bag from the second end to the first end and wherein during exhalation, exhaled gases flow into the bag from the first end to the second end, such that during both inhalation and exhalation, the bag is at least partially inflated by movement of gases through the bag to indicate breathing or respiration of a person wearing the oxygen mask.

Preferably, whereby, in use, during inhalation, gases from a surrounding atmosphere flow into the bag from the second end to the first end and wherein during exhalation, exhaled gases flow into the bag from the first end to the second end, such that during both inhalation and exhalation, the bag is at least partially inflated by movement of gases through the bag to indicate breathing or respiration of a person wearing the oxygen mask.

In another aspect, the invention provides a respiration indicating device configured to attach directly to an exhalation port of an oxygen mask, the respiration indicating device comprising a collapsible open-ended bag having a first opening at a first end and a second opening at a second end opposite the first end.

Preferably, the collapsible open-ended bag is substantially cylindrical or tubular. Preferably, the collapsible open-ended bag is conical or frustonical. Preferably, the second end of the collapsible open-ended bag is substantially rounded (or substantially hemispherical) having the second opening formed therein. Preferably, the collapsible open-ended bag is substantially arch shaped or U shaped.

Preferably, the collapsible open-ended bag comprises a substantially light-weight plastic material. Preferably, the collapsible open-ended bag comprises high density polyethylene. Preferably, the collapsible open-ended bag is coloured to provide greater visibility.

Preferably, the collapsible open-ended bag has a thickness of approximately 10 micrometres. Preferably, the collapsible open-ended bag has a thickness of no more than approximately 10 micrometres. Preferably, the collapsible open-ended bag has a thickness of no more than approximately 6 micrometres. Preferably, the collapsible open-ended bag has a thickness between 4 and 6 micrometres.

Preferably, the collapsible open-ended bag has a length of between approximately 40 millimetres and 80 millimetres. Preferably, the collapsible open-ended bag has a length of approximately 60 millimetres.

Preferably, a diameter of the first opening is greater than a diameter of the second opening. Preferably, the diameter of the first opening is between two to three times the diameter of the second opening. This ensures the collapsible open-ended bag 10 properly inflates and collapses during each stage of the breathing cycle so that proper visual indicating of breathing can be provided.

Preferably, the first opening of the collapsible open-ended bag has a diameter of between approximately 15 millimetres and 35 millimetres. Preferably, the first opening of the collapsible open-ended bag has a diameter of between approximately 20 millimetres and 35 millimetres. Preferably, the first opening of the collapsible open-ended bag has a diameter of between approximately 30 millimetres and 35 millimetres. Preferably, the first opening of the collapsible open-ended bag has a diameter of between approximately 15 millimetres and 25 millimetres. Preferably, the second opening of the collapsible open-ended bag has a diameter of between approximately 5 millimetres and 15 millimetres. Preferably, the second opening of the collapsible open-ended bag has a diameter of between approximately 10 millimetres and 15 millimetres. Preferably, the first opening of the collapsible open-ended bag has a diameter of approximately 20 millimetres. Preferably, the first opening of the collapsible open-ended bag has a diameter of approximately 33 millimetres. Preferably, the second opening of the collapsible open-ended bag has a diameter of approximately 10 millimetres.

Preferably, the collapsible open-ended bag has a volume of between approximately 5 millilitres and 10 millilitres.

Preferably, the respiration indicating device or system further comprises an attachment arrangement configured to attach the collapsible open-ended bag to an oxygen mask. Preferably, the attachment arrangement comprises an annular flange. Preferably, the annular flange comprises an annular body configured to attach or connect on a first side of the annular body to the first end of the collapsible open-ended bag and attach or connect on a second side of the annular body to the oxygen mask. Preferably, the annular flange comprises an annular body connected on a first side to the first end of the collapsible open-ended bag (such as by heat sealing or bonding, for example) and an adhesive on a second side of the annular body for attaching the annular body to the oxygen mask.

Preferably, the collapsible open-ended bag further comprises indicia thereon. Preferably, the indicia comprises a rectangular shaped indicia.

### BRIEF DESCRIPTION OF THE DRAWINGS

Preferred features, embodiments and variations of the invention may be discerned from the following Detailed Description which provides sufficient information for those skilled in the art to perform the invention. The Detailed Description is not to be regarded as limiting the scope of the preceding Summary of the Invention in any way. The Detailed Description will make reference to a number of drawings as follows:

FIG. 1 is an illustration of a collapsible open-ended bag of a respiration indicating device in accordance with an embodiment of the invention described herein;

FIG. 2 is an end view of the collapsible open-ended bag shown in FIG. 1;

FIG. 3 is an illustration of an attachment arrangement in the form of an annular flange for use in connecting the collapsible open-ended bag of FIG. 1 to an oxygen mask;

FIG. 4 is an end view of the annular flange shown in FIG. 3;

FIG. 5 illustrates the collapsible open-ended bag of FIG. 1 and the annular flange of FIG. 3 connected together forming an embodiment of a respiration indicating device;

FIG. 6 illustrates an exploded view of the respiration indicating device shown in FIG. 5, further including an adhesive ring attachable to the annular flange;

FIG. 7 illustrates a second view of the respiration indicating device shown in FIG. 5;

FIG. 8 illustrates a further view of the respiration indicating device shown in FIGS. 5 and 7;

FIG. 9 illustrates the respiration indicating device attached to an oxygen mask;

FIG. 10 illustrates a top down view of the respiration indicating device attached to the oxygen mask also shown in FIG. 9;

FIG. 11 illustrates a second view of the respiration indicating device attached to an oxygen mask;

FIG. 12 is an illustration of a collapsible open-ended bag of a respiration indicating device having indicia printed thereon in accordance with an embodiment of the invention described herein; and

FIGS. 13 to 15 illustrate a second embodiment of a collapsible open-ended bag and respiration indicating device in accordance with the present invention.

### DETAILED DESCRIPTION OF PREFERRED EMBODIMENTS

Embodiments of the invention described herein relate to a real-time respiration indicating device, method and system. The device is intended to provide a very low-cost, single-use, unpowered disposable breath or respiration indicator that does not need any ancillary equipment such as a monitor or external analysis machine. This is in contrast to existing systems which focus on measuring a time-series of data which can be analysed or displayed as a waveform graph to gain an assessment of respiratory rate, which is distinct from respiration indication and detection.

The terms “breath” and “during a breath” are used to refer to a single breath or phase of a respiratory cycle (i.e. an individual exhalation or inhalation) unless otherwise indicated.

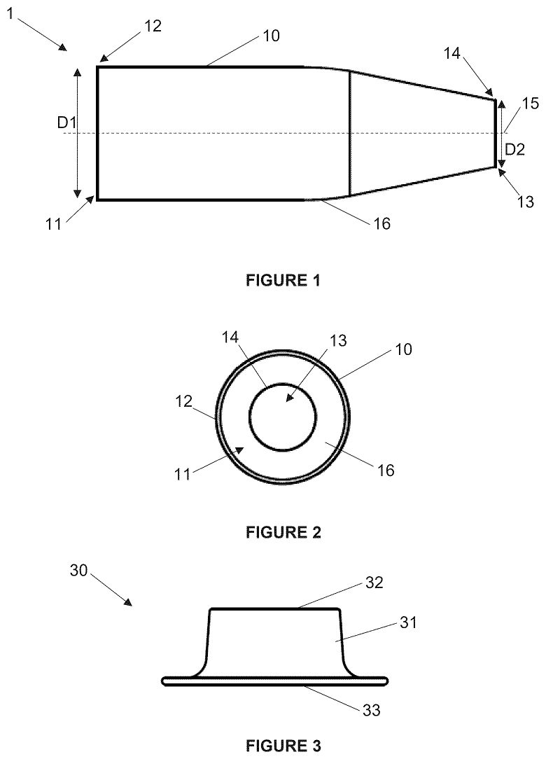

FIGS. 1 and 2 show a respiration indicating device 1 configured to attach to at least part of an exhalation port of an oxygen mask for monitoring respiration of a patient.

The respiration indicating device 1 comprises a collapsible open-ended bag 10 having a first opening 11 at a first end 12 and a second opening 13 at a second end 14 opposite the first end 12.

The first opening 11 and second opening 13 are axially aligned through a respective centre of each opening, which is represented by longitudinal axis 15 extending through the respective centres of the first opening 11 and second opening 13 in FIG. 1.

As can be seen in FIG. 1, the collapsible open-ended bag 10 has a substantially conical or frustoconical body 16, wherein the collapsible open-ended bag 10 tapers from the second end 13 to the first end 11. This shape usefully makes it easier for each breath in the respiratory cycle to be observed and monitored, particularly in the exhalation phase of the breathing cycle, as the exhaled gases are funnelled through the tapering body 16 of the bag 10.

However, it will be appreciated that in some alternative embodiments, the collapsible open-ended bag 10 may be substantially cylindrical, arch-shaped or tubular.

The collapsible open-ended bag 10 is made from a substantially light-weight plastic material, such as high density polyethylene.

The collapsible open-ended bag 10 may be coloured and/or translucent (rather than being clear or transparent) to improve visibility of the bag from a distance.

The collapsible open-ended bag 10 has a thickness of approximately 6 micrometres and preferably has a thickness of no more than approximately 10 micrometres. In some embodiments, the collapsible open-ended bag 10 has a thickness between approximately 4 micrometres and 6 micrometres. The Inventors have found that a collapsible open-ended bag 10 that is too thick and/or made from heavy materials will not move with small flows of respiratory gases.

Embodiments of the collapsible open-ended bag 10 have a length of between approximately 40 millimetres and 80 millimetres. In a preferred embodiment, the collapsible open-ended bag 10 has a length of approximately 60 millimetres.

In some embodiments, the diameter D1 of the first opening 11 is between two to three times the diameter D2 of the second opening 13. This ensures the collapsible open-ended bag 10 properly inflates and collapses during each stage of the breathing cycle so that proper visual indicating of breathing can be provided.

Furthermore, embodiments of the first opening 11 of the collapsible open-ended bag 10 have a diameter D1 of between approximately 15 millimetres and 25 millimetres, and the second opening 13 of the collapsible open-ended bag 10 has a diameter D2 of between approximately 5 millimetres and 15 millimetres. In a concial or frustoconical form, the first opening 11 of the collapsible open-ended bag 10 has a diameter D1 of approximately 20 millimetres and the second opening 13 of the collapsible open-ended bag 10 has a diameter D2 of approximately 10 millimetres.

The collapsible open-ended bag, in some embodiments, has a volume of between approximately 5 millilitres and 10 millilitres.

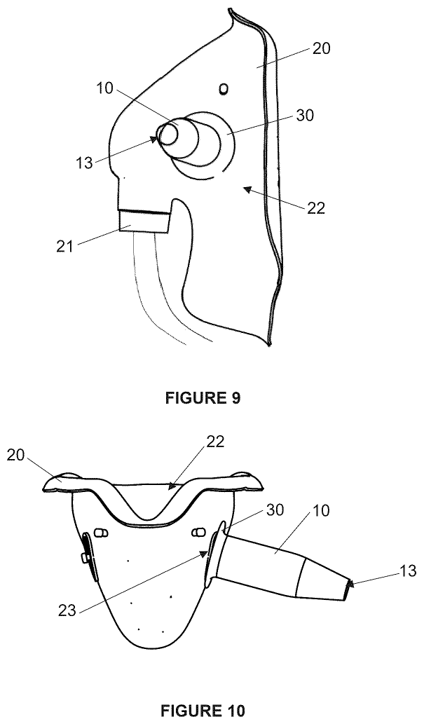

When the collapsible open-ended bag 10 is attached to an oxygen mask 20, as shown in FIGS. 9 to 11, for example, and the oxygen mask 20 is fitted to a patient (not shown) the open-ended nature of the bag 10 facilitates gases from a surrounding atmosphere to flow (or be drawn) into the bag 10 from the second end 14 to the first end 12 through the second opening 13 and first opening 11, respectively. In contrast, during exhalation, exhaled gases flow into the bag 10 from the first end 12 to the second end 14 through the first opening 11 and second opening 13, respectively. The configuration of the bag 10 is such that during both inhalation and exhalation, the bag 10 is at least partially inflated by movement of gases through the bag 10. Advantageously, the respiration indicating device 1 is functional regardless of whether oxygen is being supplied to the oxygen mask 20 through the oxygen supply spigot 21 or not.

In some embodiments, the respiration indicating device 1 further comprises an attachment arrangement 30, which can be seen in FIGS. 3 to 5, configured to attach the collapsible open-ended bag to an oxygen mask 20.

The attachment arrangement 30 is in the form of an annular flange having an annular body 31 configured to attach or connect on a first side 32 of the annular body 31 to the first end 11 of the collapsible open-ended bag 10 and attach or connect on a second side 33 of the annular body 31 to the oxygen mask 20.

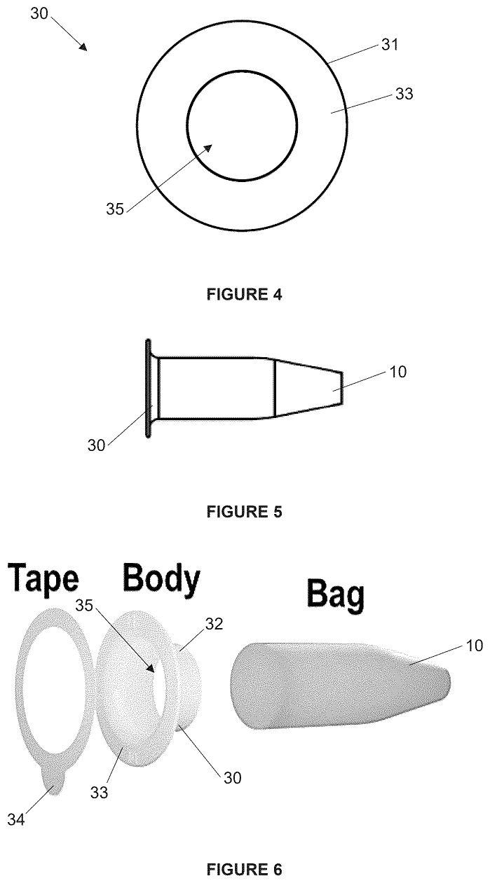

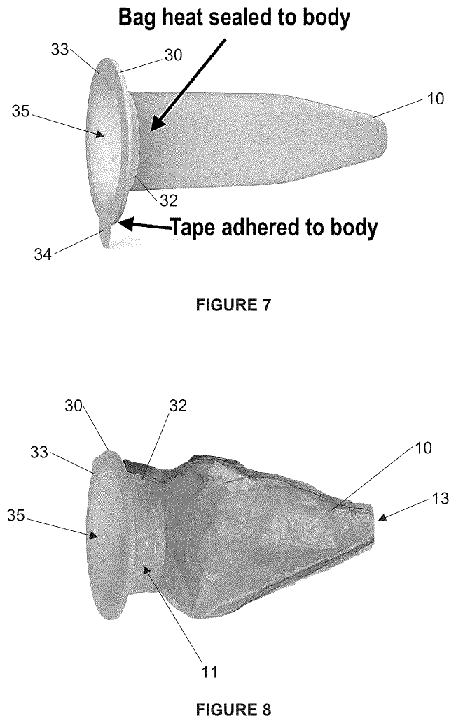

In the illustrated embodiments shown in FIGS. 6 to 8, the attachment arrangement 30 comprises an annular body 31 connected on a first side 32 to the first end 11 of the collapsible open-ended bag 10 (such as by heat sealing or bonding, for example) and an adhesive ring 34 on a second side 33 of the annular body 31 for attaching the annular body 31 to the oxygen mask 20. The collapsible open-ended bag 100 and annular body 31 can be connected through means other than heat sealing, such as an adhesive, for example. As can be seen in FIG. 4, the annular body 31 includes a passage 35 extending therethrough to allow the transit of gases between the collapsible open-ended bag 10 and the oxygen mask 20.

Turning to FIGS. 9 to 11, it will be appreciated that when the oxygen mask 20 having the collapsible open-ended bag 10 attached thereto is worn by a patient, during exhalation, gases flow from the interior 22 of the oxygen mask 20, through the exhalation port 23 and the first opening 11 to enter the interior of the bag 10 and ultimately flow out through the second opening 13. Similarly, but in reverse, during inhalation, gases flow from the surrounding atmosphere into the interior of the bag 10 through the second opening 13 and then into the interior 22 of the oxygen mask 20 through the connected first opening 11 via the passage 35 extending through the annular body 35 and exhalation portion 23. The passage 35 is preferably sized such that the annular body 31 of the annular flange completely surrounds the exhalation port 23 (i.e. the diameter of the passage 35 is equal to or greater than the diameter of the exhalation portion 23).

In some embodiments, the collapsible open-ended bag 10 attaches directly to the exhalation port 23 of the oxygen mask 20.

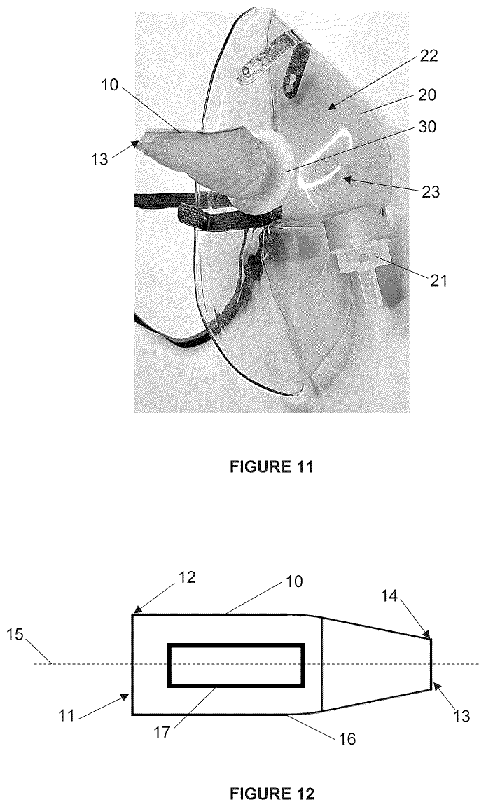

In some embodiments, the collapsible open-ended bag 10 further comprises indicia printed thereon which emphasises the inflation and deflation of the bag during the respiratory cycle and thereby provides an improved respiration indicator. In FIG. 12, the collapsible open-ended bag 10 is shown to have a rectangular indicium 17 printed thereon which acts as a visual indicator for an observer of the respiration indicating device 1. The indicia increases visibility of bag movements (i.e. deformation of lines) in the event that breathing is shallow.

It will be appreciated that the collapsible open-ended bag 10 having the rectangular indicium 17 (or any indicia) printed thereon can be used in combination with the attachment arrangement 30 and oxygen mask 20 described herein.

It will be appreciated that the indicia may take a number of different forms other than a rectangle.

In another embodiment illustrated in FIGS. 13 to 15, a respiration indicating device 1A comprises a collapsible open-ended bag 10A having a first opening 11A at a first end 12A and a second opening 13A at a second end 14A opposite the first end 12A.

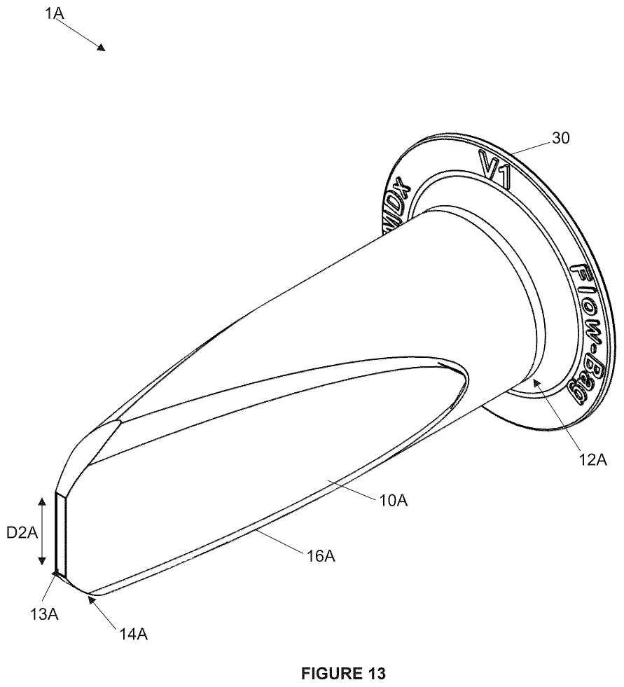

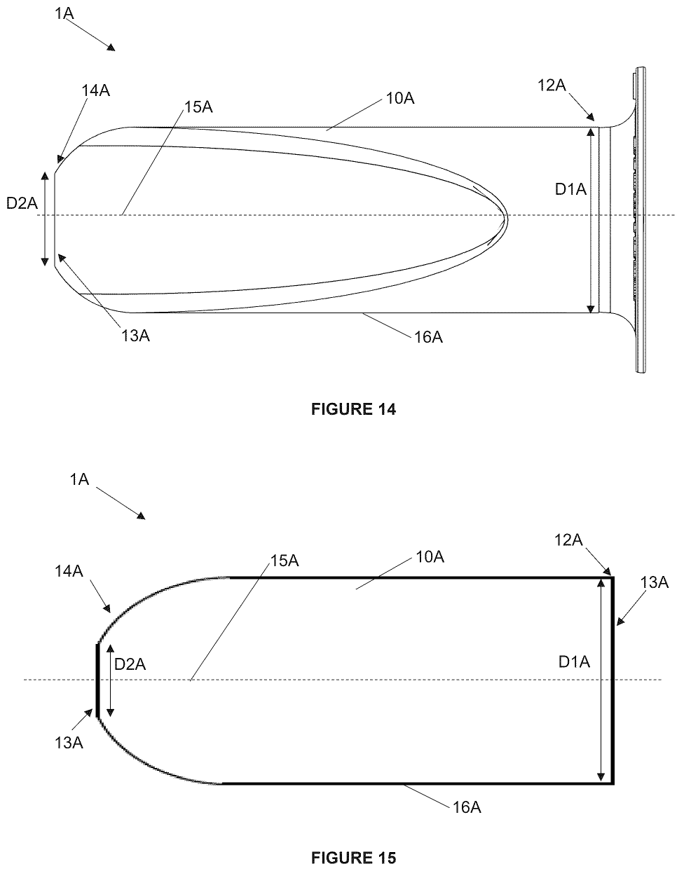

The first opening 11A and second opening 13A are axially aligned through a respective centre of each opening, which is represented by longitudinal axis 15A extending through the respective centres of the first opening 11A and second opening 13A in FIGS. 14 and 15.

As can be seen in FIGS. 13 to 15, the collapsible open-ended bag 10A has a body 16A that is substantially arch shaped or U shaped, wherein the collapsible open-ended bag 10A is rounded at the second end 13A. This shape usefully ensures that the collapsible open-ended bag 10A properly collapses and expands during each breath.

In some embodiments, the body 16A is formed from two sheets of high density polyethylene, where each sheet of high density polyethylene is arch or U shaped. The two sheets of high density polyethylene are aligned and joined or sealed along the edges leaving a first opening at a first end and a second opening at a second end in accordance with the first and second openings described herein in relation to other embodiments.

The collapsible open-ended bag 10A is made from a substantially light-weight plastic material, such as high density polyethylene.

The collapsible open-ended bag 10A may be coloured and/or translucent (rather than being clear or transparent) to improve visibility of the bag from a distance.

The collapsible open-ended bag 10A has a thickness of approximately 6 micrometres and preferably has a thickness of no more than approximately 10 micrometres. In some embodiments, the collapsible open-ended bag 10A has a thickness between approximately 4 micrometres and 6 micrometres. The Inventors have found that a collapsible open-ended bag 10A that is too thick and/or made from heavy materials will not move with small flows of respiratory gases.

Embodiments of the collapsible open-ended bag 10A have a length of between approximately 40 millimetres and 80 millimetres. In a preferred embodiment, the collapsible open-ended bag 10A has a length of approximately 60 millimetres.

A diameter D1A of the first opening 11A is greater than a diameter D2A of the second opening 13A. In some embodiments, the diameter D1A of the first opening 11A is between two to three times the diameter D2A of the second opening 13A. This ensures the collapsible open-ended bag 10 properly inflates and collapses during each stage of the breathing cycle so that proper visual indicating of breathing can be provided.

Furthermore, embodiments of the first opening 11A of the collapsible open-ended bag 10A have a diameter D1A of between approximately 15 millimetres and 35 millimetres, and the second opening 13 of the collapsible open-ended bag 10 has a diameter D2A of between approximately 5 millimetres and 15 millimetres.

In some embodiments, the first opening 11A of the collapsible open-ended bag 10A has a diameter D1A of between approximately 30 millimetres and 35 millimetres and the second opening 13A of the collapsible open-ended bag 10A has a diameter D2A of between approximately 10 millimetres and 15 millimetres.

In another embodiment, the first opening 11A of the collapsible open-ended bag 10A has a diameter D1A of approximately 33 millimetres and the second opening 13A of the collapsible open-ended bag 10A has a diameter D2A of approximately 10 millimetres.

The collapsible open-ended bag 10A, in some embodiments, has a volume of between approximately 5 millilitres and 10 millilitres.

The collapsible open-ended bag 10A can be connected to or attached to an attachment arrangement 30 as described above. The attachment arrangement 30 may take the form of an annular flange as described herein in relation to other embodiments.

When the collapsible open-ended bag 10A is attached to an oxygen mask, as shown in FIGS. 9 to 11 in relation to another embodiment of the collapsible open-ended bag, for example, and the oxygen mask is fitted to a patient (not shown) the open-ended nature of the bag 10A facilitates gases from a surrounding atmosphere to flow (or be drawn) into the bag 10A from the second end 14A to the first end 12A through the second opening 13A and first opening 11A, respectively. In contrast, during exhalation, exhaled gases flow into the bag 10A from the first end 12A to the second end 14A through the first opening 11A and second opening 13A, respectively. The configuration of the bag 10A is such that during both inhalation and exhalation, the bag 10A is at least partially inflated by movement of gases through the bag 10A. Advantageously, the respiration indicating device 1A is functional regardless of whether oxygen is being supplied to the oxygen mask through an oxygen supply spigot or not.

In use, embodiments of the invention include attaching a collapsible open-ended bag having a first opening at a first end and a second opening at a second end opposite the first end to an oxygen mask, wherein the first opening at least partially covers an exhalation port of the oxygen mask. Subsequently, during inhalation, gases from a surrounding atmosphere flow into the bag from the second end to the first end. Conversely, during exhalation, exhaled gases flow from the oxygen mask into the bag from the first end to the second end, such that during both inhalation and exhalation, the bag is at least partially inflated by movement of gases through the bag to thereby indicate breathing or respiration of a patient.

The respiration indicating device described herein can be used to indicate inhalation and exhalation of a patient (i.e. breathing or respiration), change in depth of breath and change in rate of breath.

As noted above, breath or respiration detection occurs with exhalation of respiratory gases that exit the oxygen mask side exhalation port and flows through the bag, and visa-versa for inhalation with room air flowing through the bag and into the mask. Thus, the respiration indicating device described herein does not require oxygen to be flowing at all in order to function.

Advantageously, embodiments of the invention utilise exhalation ports on a standard oxygen mask to maintain fluid communication with respiratory gases.

Embodiments of the invention disclosed herein are adhered to a standard oxygen mask's side exhalation port with an adhesive and thus the oxygen mask does not require any modifications in order to function with the respiration indicating device.

In compliance with the statute, the invention has been described in language more or less specific to structural or methodical features. The term “comprises” and its variations, such as “comprising” and “comprised of” is used throughout in an inclusive sense and not to the exclusion of any additional features.

It is to be understood that the invention is not limited to specific features shown or described since the means herein described comprises preferred forms of putting the invention into effect.

The invention is, therefore, claimed in any of its forms or modifications within the proper scope of the appended claims appropriately interpreted by those skilled in the art.

1. A respiration indicating device configured to attach to an exhalation port of an oxygen mask, the respiration indicating device comprising:
a collapsible open-ended bag having a first opening at a first end and a second opening at a second end opposite the first end,
wherein during inhalation, gases from a surrounding atmosphere flow into the bag from the second end to the first end and wherein during exhalation, exhaled gases flow into the bag from the first end to the second end, such that during both inhalation and exhalation, the bag is at least partially inflated by movement of gases through the bag.

2. The respiration indicating device of claim 1, wherein the collapsible open-ended bag is substantially cylindrical, tubular, arch shaped or U shaped.

3. The respiration indicating device of claim 2, wherein the second end of the collapsible open-ended bag is substantially rounded having the second opening formed therein.

4. The respiration indicating device of claim 1, wherein a diameter of the first opening is greater than a diameter of the second opening.

5. The respiration indicating device of claim 1, wherein a diameter of the first opening is between two to three times a diameter of the second opening.

6. The respiration indicating device of claim 1, wherein the first opening of the collapsible open-ended bag has a diameter of approximately 33 millimetres and the second opening of the collapsible open-ended bag has a diameter of approximately 10 millimetres.

7. The respiration indicating device of claim 1, wherein the collapsible open-ended bag is coloured to provide greater visibility.

8. The respiration indicating device of claim 1, wherein the collapsible open-ended bag has a thickness of approximately 10 micrometres and the thickness is no more than approximately 10 micrometres.

9. The respiration indicating device of claim 1, wherein the collapsible open-ended bag has a thickness of no more than approximately 6 micrometres.

10. The respiration indicating device of claim 1, wherein the collapsible open-ended bag has a thickness between 4 and 6 micrometres.

11. The respiration indicating device of claim 1, wherein the collapsible open-ended bag has a length of between approximately 40 millimetres and 80 millimetres.

12. The respiration indicating device of claim 1, wherein the collapsible open-ended bag is configured to attach to an exhalation port of an oxygen mask.

13. The respiration indicating device of claim 1, whereby, in use, during inhalation, gases from a surrounding atmosphere flow into the bag from the second end to the first end and wherein during exhalation, exhaled gases flow into the bag from the first end to the second end, such that during both inhalation and exhalation, the bag is at least partially inflated by movement of gases through the bag to indicate breathing or respiration of a person wearing the oxygen mask.

14. The respiration indicating device of claim 1, wherein the respiration indicating device further comprises an attachment arrangement configured to attach the collapsible open-ended bag to an oxygen mask.

15. The respiration indicating device of claim 14, wherein the attachment arrangement comprises an annular flange and wherein the annular flange comprises an annular body connected on a first side to the first end of the collapsible open-ended bag and an adhesive on a second side of the annular body for attaching the annular body to the oxygen mask.

16. The respiration indicating device of claim 1, wherein the collapsible open-ended bag comprises high density polyethylene.

17. A method for indicating respiration, the method comprising:
attaching a collapsible open-ended bag having a first opening at a first end and a second opening at a second end opposite the first end to an oxygen mask, wherein the first opening at least partially covers an exhalation port of the oxygen mask;
during inhalation, gases from a surrounding atmosphere flow into the bag from the second end to the first end; and
during exhalation, exhaled gases flow from the oxygen mask into the bag from the first end to the second end, such that during both inhalation and exhalation, the bag is at least partially inflated by movement of gases through the bag.

18. A respiration indicating system comprising:
an oxygen mask having an exhalation port;
a collapsible open-ended bag having a first opening at a first end and a second opening at a second end opposite the first end, the first end attached to the oxygen mask over a ventilation port such that an interior of the bag receives exhaled gases from within the oxygen mask,
wherein, in use, during inhalation, gases from a surrounding atmosphere flow into the bag from the second end to the first end and wherein during exhalation, exhaled gases flow into the bag from the first end to the second end, such that during both inhalation and exhalation, the bag is at least partially inflated by movement of gases through the bag to indicate breathing or respiration of a person wearing the oxygen mask.

19. A respiration indicating device configured to attach directly to an exhalation port of an oxygen mask, the respiration indicating device comprising a collapsible open-ended bag having a first opening at a first end and a second opening at a second end opposite the first end.
